(19) United States
(12) Patent Application Publication
(10) Pub. No.: US 2026/0145943 A1
(43) Pub. Date: May 28, 2026

(54) METHOD FOR GENERATING THERMAL AND/OR ELECTRICAL ENERGY

(71) Applicant: OBRIST TECHNOLOGIES GMBH, Lustenau (AT)

(72) Inventor: Frank OBRIST, Bregenz (AT)

(21) Appl. No.: 19/122,187

(22) PCT Filed: Sep. 12, 2023
(86) PCT No.: PCT/EP2023/075023

§ 371 Date: Apr. 17, 2025

(30) Foreign Application Priority Data
Oct. 19, 2022 (DE) 102022127420.9

(51) Int. Cl. C01B 32/05; C07C 1/12; C07C 29/151; C25B 1/04; C25B 15/08

(52) U.S. Cl. CPC C01B 32/05; C07C 1/12; C07C 29/1518; C25B 1/04; C25B 15/081

(57) ABSTRACT

A method is for the environmentally friendly generation of thermal and/or electrical energy using a CO2-negative liquid fuel for combustion in a stationary thermal power plant. Thermal energy is harnessed from the combustion of the liquid fuel and/or converted into electrical energy. Liquid fuel is produced in an atmospheric carbon-dioxide-reducing process, which is supplied with current from, in particular exclusively, at least one renewable energy source.

### CROSS-REFERENCE TO RELATED APPLICATIONS

The present application is a U.S. National Stage of International Patent Application PCT/EP2023/075023 filed Sep. 12, 2023 and claims priority to DE Patent Application No. 102022127420.9 filed Oct. 19, 2022, both of which applications are incorporated herein by reference to the extent appropriate.

### DESCRIPTION

The invention relates to a method for generating thermal and/or electrical energy.

Since the beginning of the industrial revolution in 1800, the CO₂ concentration in the atmosphere has risen from previously stable 280 ppmv (parts per million by volume) to 410 ppmv in the year 2020. It is predicted that this rise will continue or respectively further intensify if no technologies are used to curb the carbon emissions.

The ratified Paris Agreement names as its principal aim to keep the rise of the global average temperature below 2° C. over the preindustrial level, which requires a reduction of CO₂ emissions to zero by 2050. The proposals for limiting these emissions include the use of biofuels, solar energy and wind turbines. The reduction of the previous CO₂ emissions and hence the limiting of the rise of the CO₂ proportion in the atmosphere is, however, not sufficient in the long term in order to rectify the imbalance between oxygen and CO₂ in the atmosphere, which has resulted through the hitherto overproduction of CO_{2.}

The present energy supply network, by which in particular electric current and/or district heating is distributed, is based—in addition to energy generation through nuclear fuels—principally on the combustion of fossil fuels, in particular natural gas and coal. This is accompanied by high CO₂ emissions. CO₂ emissions were indeed already reduced through power-heat coupling, in particular also in the combustion of refuse as fuel. However, for a more environmentally friendly, in particular climate-Substitute neutral or respectively CO₂-neutral, energy supply, a global approach is expedient, which has in view all processes for energy generation and in particular also takes into consideration the production of the fuel which is used.

The object of the invention consequently consists in indicating a method for generating energy with the aim of a considerably improved CO₂ balance of the worldwide energy supply.

The invention solves the above-mentioned problem by a method for generating electrical and/or thermal energy with the following steps:
Producing a liquid fuel in an atmospheric carbon-dioxide-reducing process, which is supplied with current from, in particular exclusively, at least one renewable energy source and comprises the following steps:
a) Producing oxygen in an electrolysis unit, which through at least one water supply line receives a quantity of water, in particular from the sea, and breaks down the received quantity of water into an oxygen partial quantity and a hydrogen partial quantity;
b) Directing a first part of the hydrogen partial quantity from the electrolysis unit to a carbonization unit, and a second part of the hydrogen partial quantity to a fuel synthesis unit;
c) Cleaning ambient air in at least one carbon dioxide sorption unit, wherein the carbon dioxide sorption unit receives the ambient air through at least one air inlet and extracts a carbon dioxide quantity from the ambient air in at least one downstream sorber facility;
d) Directing a first part of the carbon dioxide quantity to the carbonization unit, and a second part of the carbon dioxide quantity to the fuel synthesis unit;
e) Producing carbon in the carbonization unit, in particular through methane synthesis and methane splitting and/or through methanol synthesis and methanol splitting, and
f) Combining the second part of the hydrogen partial quantity and the second part of the carbon dioxide quantity in the fuel synthesis unit to produce the liquid fuel, and
Using the liquid fuel for the generation of a quantity of thermal energy and/or a quantity of electrical energy in a stationary thermal power plant.

The invention makes it possible to generate thermal and/or electrical energy not only in a CO₂-neutral manner but even in a CO₂-reducing or respectively CO₂-negative manner. Through the sorption of CO₂ from the ambient air, the CO₂ proportion in the earth's atmosphere is reduced and thus the constant rise of CO₂ pollution in the last decades and centuries is successively reversed. The invention enables at the same time the production of pure oxygen and also reduces in this respect the CO₂ proportion in the earth's atmosphere. The liquid fuel which is used in the invention is consequently CO₂-negative, i.e. in the production of the liquid fuel, more CO₂ is extracted from the earth's atmosphere than is released again through its later combustion.

Preferably, the negative proportion in the CO₂ balance of the method according to the invention is at least 5%, in particular at least 3%, in particular at least 10%, in particular at least 20%, in particular at least 30%, in particular at least 50%, in particular at least 100%, in particular at least 200%. It is expected that the current climate change, produced through industrial development, is braked in the short term, stopped in the medium term and reversed in the long term, through a worldwide comprehensive deployment of the invention.

In order to actively reduce the CO₂ proportion in the atmosphere, the carbon which is produced in the carbonization unit is preferably delivered to a carbon store. The carbon store can be, in particular, a sea or respectively a seabed. In other words, the carbon, in particular in the form of graphite, can be stored permanently on the seabed. In this respect, in a preferred embodiment of the invention provision is made that step e) of the process for producing the liquid fuel comprises the transporting of the carbon, in particular to a long-term carbon store, in particular to a region of the seabed.

The stationary thermal power plant can be a turbine-driven power plant and/or a motor-operated power plant and/or a power-heat coupling plant and/or a heating plant.

It is advantageous if the liquid fuel is produced in a production system which is separated spatially from the stationary thermal power plant. The separation of the fuel production from the fuel use makes it possible, on the one hand, to carry out the production of the liquid fuel where the energy which is necessary for the production, preferably generated in a renewable manner, is available. This may be, for example, in the vicinity of an offshore wind farm or of a photovoltaic system, wherein these may be situated at locations in the world which enable a high efficiency of renewal energy generation. This may be regions, for example, which have a high and constant wind or a high solar radiation.

On the other hand, the stationary thermal power plant can be established where the thermal and/or electrical energy generated in the power plant is required, for example in the vicinity of an industrial production system or a settlement. The distance between the production system and the stationary thermal power plant may be at least 50 km, in particular at least 100 km, preferably at least 500 km.

In particular in the case of fuel production and fuel use which are spatially separated from one another, provision may be preferably made that the liquid fuel can be transported by at least one transport system, in particular a pipeline or a tanker vehicle, to the stationary thermal power plant.

In a particularly preferred configuration of the invention, the production system comprises a photovoltaic system as renewable energy source. The photovoltaic system can be situated in a region with a global horizontal solar radiation per year of at least 1,500 kWh/m², in particular 2000 kWh/m². The problem of the increasing CO₂ emissions is global and therefore global efforts are necessary in order to solve this problem and to save the global climate. It is therefore desirable to operate the method according to the invention efficiently on a large scale. The use of photovoltaics as renewable energy source has the advantage that the regions with high global horizontal solar radiation often also have access to the sea. Production systems can therefore be operated very efficiently for the production of the liquid fuel necessary for the invention, because all the necessary starting components, solar energy, CO₂ and water, are available at a short distance. In addition, the energy generation by means of a photovoltaic system is very cost-effective. Compared to other technologies for renewable energy generation, the energy generation by means of photovoltaics is three to ten times more cost-effective. This applies in particular when the method is carried out in a production system which is situated in a region with a high duration of sunshine hours or high global horizontal solar radiation, for example in Saudi Arabia.

The extraction of the carbon component from the atmosphere can take place through a two-stage process, for example through a methane synthesis, followed by a methane splitting. In the methane synthesis, the hydrogen which is delivered to the carbonization unit is converted with the likewise delivered carbon into methane, which is subsequently separated again through the methane splitting. A Kvaerner process can be used here. Alternatively, the methane splitting can comprise a methane pyrolysis process, designated as a monolith process. Advantageously, a methane splitting method is used in which the split-off carbon is issued as solid material. This is fulfilled for example in a Kvaerner process.

Alternatively or additionally, provision can further be made to produce the carbon through methanol synthesis and methanol splitting.

The (waste) heat occurring at the carbonization in the carbonization unit can be directed to the carbon dioxide sorption unit and utilized there as energy for the carbon sorption. Additionally or alternatively, the (waste) heat can be directed from the fuel synthesis unit to the carbon dioxide sorption unit and utilized there as energy for the carbon sorption. The efficiency of the method overall is thereby further increased, and the primary energy requirement of the method is reduced.

Preferably, the oxygen partial quantity and the cleaned ambient air are emitted to the external atmosphere, and the hydrogen partial quantity and the carbon dioxide quantity in the carbonization unit are converted into water, carbon and heat. This enables a reduction of the carbon dioxide proportion in the atmospheric air and hence the balancing of an existing imbalance of the quantities of the air components.

The invention is explained more closely below with the aid of further details with reference to the enclosed drawings.

The single FIGURE shows therein a perspective view of a production system for the production of a CO₂-negative liquid fuel.

The invention comprises substantially two steps, on the one hand the production of a CO₂-negative liquid fuel in a production system, and on the other hand the use of this liquid fuel in a stationary thermal power plant for the generation of thermal and/or electrical energy.

The production of the CO₂-neutral liquid fuel takes place preferably in a production system 10, which is situated in an area with high global horizontal solar radiation and in the vicinity of the sea, for example in Saudi Arabia. The production system 10 preferably concerns a large power plant. The production system 10 can have at least one installation area 18 which is connected to a foundation of a building and/or of a structure.

The production system 10 can comprise an electrolysis unit 11 for the production of oxygen and a carbon dioxide sorption unit 12 for cleaning the ambient air UL of the external atmosphere surrounding the production system 10. Generally it is possible that the electrolysis unit 11 and/or the carbon dioxide sorption unit 12 are arranged in a shared building or in separate buildings. The production system 10 can further comprise a power generation unit 31 for the self-sufficient current supply of the production system 10, which will be elaborated on later.

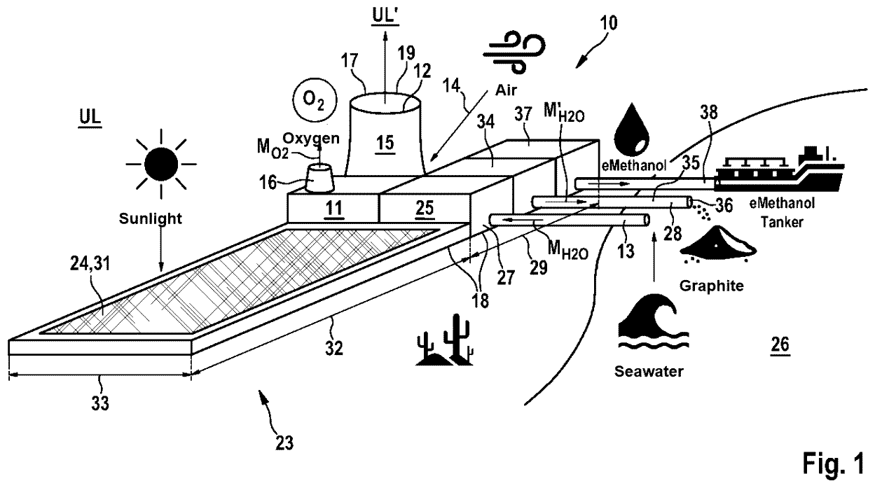

The electrolysis unit 11 is configured to receive a water quantity M_{H2O} through electrolysis into an oxygen partial quantity M_{O2} and a hydrogen partial quantity. The electrolysis unit 11 thus forms a unit for water electrolysis. The electrolysis unit 11 is connected to a water supply line 13 to receive the water quantity M_{H2O}. As can be seen in FIG. 1, a pump unit 25 is arranged between the electrolysis unit 11 and the water supply line 13. The pump unit 25 has at least one pump for the conveying of water from a water reservoir 26. The water reservoir 26 can be a sea with seawater.

In order to prepare the seawater for the electrolysis process via the electrolysis unit 11, the production system 10 can have a seawater desalination unit 27. The seawater desalination unit 27 is adapted to separate out a particular salt proportion from the conveyed seawater quantity M_{H2O}, so that after the desalination process by the seawater desalination unit 27, the seawater has a reduced salt content. The desalinated seawater quantity M_{H2O} corresponds to the water quantity M_{H2O} which is broken down through the electrolysis unit 11 into an oxygen partial quantity M_{O2} and a hydrogen partial quantity. The electrolysis unit 11 is connected to the seawater desalination unit 27 by at least one pipeline. For the emission of the produced oxygen partial quantity M_{O2}, the electrolysis unit 11 has an oxygen outlet 16, which opens out into the external atmosphere. It is possible that the electrolysis unit 11 has one or more oxygen outlets 16 for the emission of the produced oxygen partial quantity M_{O2}.

The production system 10 has, furthermore, at least one (not illustrated) hydrogen transport facility, which is adapted to make available a first part of the hydrogen partial quantity, separated off from the water quantity M_{H2O}, to a carbonization unit 34 for further processing. A second part of the hydrogen partial quantity can be delivered to a fuel synthesis unit 37, for example a methanol synthesis unit.

According to FIG. 1, the carbon dioxide sorption unit 12 has an air inlet 14 for the delivery of the ambient air UL and a downstream sorber facility 15. It is possible that that carbon dioxide sorption unit 12 has one or more air inlets 14. The sorber facility 15 is connected to the air inlet 14. The sorber facility 15 is adapted to extract a carbon dioxide quantity from the ambient air UL. The carbon dioxide sorption unit 12 has, furthermore, an air outlet 17. The air outlet 17 serves for the delivery of the ambient air UL′ which is cleaned of carbon dioxide. The air outlet 17 can be aligned in vertical direction upwards and/or be part of a chimney 19.

Specifically, the sorber facility 15 is arranged between the air inlet 14 and the air outlet 17. In operation, the ambient air UL flows through the air inlet 15 to the sorber facility 15, which separates off, in particular filters, a specific carbon dioxide quantity from the air UL, wherein the cleaned ambient air UL′ flows after the sorber facility 15 through the air outlet 17 into the external atmosphere.

The production system 10 comprises furthermore a carbon dioxide transport facility, which is configured to make available the carbon dioxide quantity, separated off from the ambient air UL, to a carbon dioxide intermediate store and/or to the carbonization unit 34 of the production system 10 for further processing. Preferably, the first part of the hydrogen partial quantity and the first part of the carbon dioxide quantity are thus delivered to the carbonization unit 34, so that the extracted carbon dioxide quantity with the separated-off hydrogen partial quantity is processed to further intermediate- or end products. Specifically, the first part of the carbon dioxide quantity and the second part of the hydrogen partial quantity can be converted into water, carbon (graphite) and heat through the methanation which is carried out in the carbonization unit 34.

As is shown in FIG. 1, the production system 10 has a planar system area 23. The planar system area 23 preferably directly adjoins the electrolysis unit 11. A power generation unit 31, which is a photovoltaic system 24, is arranged on the planar system area 23. The photovoltaic system 24 is connected to the respective units of the production system 10 for the current supply. The photovoltaic system 24 is adapted in such a way that the entire production system 10 is able to be operated in a self-sufficient manner in terms of energy. This is to be understood to mean that the electric current for operating the entire production system 10 can be provided exclusively by solar energy by means of the photovoltaic system 24. In other words, preferably no fossil energy sources are used for the operation of the production system 10. The power generation unit 31 preferably comprises an energy store (not illustrated), which is adapted to supply the production system 10 with current during nighttime operation. Alternatively to the photovoltaic system 24, other units can also be used for the generation of renewable electrical energy, for example wind energy plants, in particular offshore wind farms.

The seawater desalination unit 27 described above is connected to a water return line 28, through which a seawater quantity M′_{H2O} with increased salt content, which is to be returned, is returned into the sea. Specifically, from the removed seawater quantity a particular salt content is extracted and subsequently returned into the sea again with a portion of the removed seawater quantity as water quantity M′_{H2O} which is to be returned. Thereby, a water cycle is provided, which is harmless to nature.

The production system 10 further comprises a fuel synthesis unit 37. The fuel synthesis unit 37 is connected to the electrolysis unit 11 or to a hydrogen intermediate store through a hydrogen transport facility and to the carbon dioxide sorption unit 12 through a carbon dioxide transport facility. From the delivered hydrogen and carbon, the fuel synthesis unit 37 synthesises a liquid fuel, preferably methanol, which can be removed from the production system 10 via a fuel outlet 38. The fuel can be distributed worldwide to decentralized fuel stores in particular by means of a fuel distribution system, which can comprise pipelines, ships, in particular tankers, tank freight trains and/or tank trucks. The fuel stores can be connected in particular to stationary thermal power plants, in order to make the fuel available there for the operation of the respective plant.

Through a corresponding control of the method in the production system 10, an adjustment can be made as to which proportion of the carbon, which is sorbed in the carbon dioxide sorption unit, is utilized for the production of the liquid fuel or for the production of graphite for storage in a carbon store. Initially, it is likely that a ratio of 20% graphite and 80% liquid fuel will be expedient, wherein the proportion of liquid fuel can be reduced successively over the course, and the proportion of graphite can be increased, when the requirement for the production of liquid fuel falls, in particular through the construction of further production systems 10.

The method for operating the production system 10 and hence for the producing of CO₂-negative fuel is described more closely below.

In a first method step, a water quantity M_{H2O} is received by means of the electrolysis unit 11 for the oxygen production through the water supply line 13. The received water quantity M_{H2O} is subsequently broken down through an electrolysis process into an oxygen partial quantity M_{O2} and a hydrogen partial quantity. The hydrogen partial quantity is made available through at least one hydrogen transport facility to a carbonization unit 34 for further processing, wherein the carbonization unit 34 in the present example embodiment brings about a methanation process, which comprises a methane synthesis and a methane splitting.

In a second method step, ambient air UL of an external atmosphere surrounding the production system 10 is cleaned by the carbon dioxide sorption unit 12. The ambient air UL is introduced, in particular sucked in, through several air inlets 14 into the flow channels 21, and is delivered to the downstream sorber facilities 15. Subsequently, the sorber facilities 15 extract a carbon dioxide quantity from the delivered ambient air UL. A first part of the carbon dioxide quantity is delivered through the carbon dioxide transport facility to the carbonization unit 34 for methanation. Subsequently, the obtained oxygen partial quantity M_{O2}, after the breaking-down process, and the cleaned ambient air UL', after the extraction of the carbon dioxide quantity, is emitted into the external atmosphere. Thereby, the oxygen proportion in the air is increased, and the CO₂ proportion in the air is reduced.

The first part of the hydrogen partial quantity is further converted, together with the first part of the carbon dioxide quantity, into water, carbon or respectively graphite, and heat by means of the methanation process.

In the method, seawater is desalinated, and the desalinated seawater is subsequently split by means of electrolysis into hydrogen and oxygen. The oxygen O₂ is emitted to the ambient air, in particular into the atmosphere, so that the oxygen proportion in the environment of the production system is increased. Parallel thereto, carbon dioxide CO₂ is collected by means of a carbon dioxide sorption from the ambient air UL, in particular from the atmosphere. As with the electrolytically produced hydrogen or respectively the hydrogen partial quantity, the first part of the carbon dioxide quantity removed from the ambient air UL is directed to the carbonization unit 34.

The carbon or respectively graphite can be subsequently delivered via the carbon transport facility 35 to a carbon store. The carbon store can be, for example, the water reservoir 26 or respectively the sea. As the graphite occurring in the methanation process has hardly to any impurities and is solidified similar to rock, there are no objections to dumping the graphite in the sea.

Alongside the carbon-reducing process mentioned above, a second part of the hydrogen partial quantity and a second part of the carbon dioxide quantity are delivered to the fuel synthesis unit 37 and are combined there for the production of the CO₂-negative liquid fuel. Waste products, such as hydrogen and/or oxygen, from the methanation process can be used for the fuel synthesis.

The energy required for the electrolysis, the carbon dioxide sorption and the methanation originates from renewable energy sources, specifically from the photovoltaic system 24, so that no additional production of carbon dioxide takes place here.

Through the method described here, it is therefore possible to remove carbon dioxide efficiently from the earth's atmosphere, and to break it down into its components graphite and oxygen, while at the same time a liquid fuel is produced, which has fewer effects on climate change than any known fossil fuel. The oxygen can be returned into the atmosphere, and the graphite can be stored permanently in a carbon store, for example the sea. In the production of the CO₂-negative liquid fuel, CO₂ is removed from the atmospheric air, and the excess carbon is stored in a carbon store. In this way, the method efficiently achieves an improvement to the atmospheric air quality.

### LIST OF REFERENCE NUMBERS

10 production system
11 electrolysis unit
12 carbon dioxide sorption unit
13 water supply line
14 air inlet
15 sorber facility
16 oxygen outlet
17 air outlet
18 installation area
19 chimney
23 planar system area
24 photovoltaic system
25 pump unit
26 water reservoir
27 seawater desalination unit
28 water return line
29 partial longitudinal extent
31 power generation unit
32 longitudinal extent
33 transverse extent
34 carbonization unit
35 carbon transport facility
36 carbon outlet
37 fuel synthesis unit
38 fuel outlet
UL ambient air
UL′ cleaned ambient air
M_{H2O} removed water quantity
M′_{H2O} returned water quantity
M_{O2} oxygen partial quantity

1. A method for generating electrical and/or thermal energy with the following steps:
Producing a CO₂-negative liquid fuel in an atmospheric carbon-dioxide-reducing process, which is supplied with current from at least one renewable energy source and comprises the following steps:
a) Producing oxygen in an electrolysis unit which through at least one water supply line receives a quantity of water, and breaks down the received quantity of water into an oxygen partial quantity and a hydrogen partial quantity;
b) Directing a first part of the hydrogen partial quantity from the electrolysis unit to a carbonization unit, and a second part of the hydrogen partial quantity to a fuel synthesis unit;
c) Cleaning ambient air in at least one carbon dioxide sorption unit, wherein the carbon dioxide sorption unit receives the ambient air through at least one air inlet and extracts a carbon dioxide quantity from the ambient air in at least one downstream sorber facility;
d) Directing a first part of the carbon dioxide quantity to the carbonization unit, and a second part of the carbon dioxide quantity to the fuel synthesis unit;
e) Producing carbon in the carbonization unit, and
f) Combining the second part of the hydrogen partial quantity and the second part of the carbon dioxide quantity in the fuel synthesis unit to produce the CO₂-negative liquid fuel, and
Using the CO₂-negative liquid fuel for the generation of a quantity of thermal energy and/or a quantity of electrical energy in a stationary thermal and/or electric power plant.

2. The method according to claim 1, wherein
step e) comprises transporting of the carbon.

3. The method according to claim 1, wherein
the stationary thermal and/or electric power plant is a turbine-driven power plant and/or a motor-operated power plant and/or a power-heat coupling plant and/or a heating plant.

4. The method according to claim 1, wherein
the CO₂-negative liquid fuel is produced in a production system which is separated spatially from the stationary thermal and/or electric power plant.

5. The method according to claim 4, wherein
the CO₂-negative liquid fuel is transported by at least one transport system, from the production system to the stationary thermal and/or electric power plant.

6. The method according to claim 4, wherein
the production system comprises a photovoltaic system as a renewable energy source and is situated in a region with a global horizontal solar radiation per year of at least 1,500 kWh/m².

7. The method according to claim 1, wherein the current is supplied exclusively from the at least one renewable energy source.

8. The method according to claim 1, wherein the quantity of water is exclusively from the sea.

9. The method according to claim 1, wherein the producing carbon in the carbonization unit comprises methane synthesis and methane splitting and/or methanol synthesis and methanol splitting.

10. The method according to claim 2, wherein the carbon is transported to a long-term carbon store.

11. The method according to claim 2, wherein the carbon is transported to a region of the seabed.

12. The method according to claim 5, wherein the at least one transport system comprises a pipeline or a tanker vehicle.
